(19) United States
(12) Patent Application Publication
(10) Pub. No.: US 2026/0224255 A1
(43) Pub. Date: Aug. 6, 2026

(54) MEDICAL DEVICE FOR REGENERATING BONE AND A METHOD OF MANUFACTURE THEREOF

(71) Applicant: PBC Innovations Limited, Shannon, Co. Clare (IE)

(72) Inventors: Gerard INSLEY, Shannon, Co. Clare (IE); Robert FLAVIN, Shannon, Co. Clare (IE); Ivana IVANKOVIC, Shannon, Co. Clare (IE)

(21) Appl. No.: 19/152,108

(22) PCT Filed: Jan. 26, 2024
(86) PCT No.: PCT/EP2024/052001

§ 371 Date: Jul. 30, 2025

Related U.S. Application Data
(60) Provisional application No. 63/442,143, filed on Jan. 31, 2023.

(51) Int. Cl. A61B 17/80; A61B 17/00; A61L 27/04; A61L 27/32

(52) U.S. Cl. CPC A61B 17/8095; A61L 27/047; A61L 27/32; A61B 2017/00004; A61B 2017/00526; A61L 2420/02; A61L 2430/02

(57) ABSTRACT

Device for regenerating bone, the device being formed from magnesium or an alloy of magnesium, and including a coating layer of Calcium phosphate and the Calcium Phosphate is not hydroxyapatite (HA).

### FIELD OF THE INVENTION

The present invention relates to a medical device and to a method of manufacture thereof.

In a preferred embodiment, the device is manufactured from an octacalcium phosphate (OCP) coated magnesium alloy base. The OCP magnesium alloy devices function to promote bone healing in an effective manner, whilst maintaining implant stability during the healing process. The magnesium alloy device is highly biocompatible, and is designed to degrade in vivo surroundings, wherein the rate of degradation is managed by the thickness of the coating of OCP.

In a preferred embodiment, the device may be provided in the form of a wedge.

### BACKGROUND OF THE INVENTION

Joint correction is a common procedure carried out in orthopaedic medicine. It is required across populations, usually as a result of post-traumatic deformities, ankle deformity secondary to systemic illnesses, idiopathic ankle arthritis, and for paediatric deformity correction.

Osteotomy is the typical surgical practice to realign a misaligned joint. This is carried out by distraction of the related bone or bones into position for realignment. The joint is held in alignment usually with a plate and screw fixing, a wedge insertion, or potentially both. Current issues with plate and screw fixations include dislodgement of bone fragments with loosening screws/plates.

Furthermore, the insertion of plates require large incisions areas and can lead to wound complications due to poor blood supply, poor quality skin and anatomical location (such as distal tibia). Due to the thin skin, joint pain and discomfort can be felt by patients. This is especially true of elderly patients.

Wedges, on the other hand are inserted into the distraction site and are used to support the alignment of the joint. Wedges induce fewer problems with later wound complications, however they have tendencies to mislocate and/or dislodge, ultimately leading to pain and need for re-surgery. In addition, wedges according to the art are intended to remain as a permanent foreign body in the bone, with potential future complications such as infections, stiffness, and material breakage.

Therefore, there is a need to address these clinical needs, by minimising the invasivity of the surgical process, prevent dislodgment and movement of the device and to avoid the use of a permanent foreign object in the body.

It is therefore an object of at least one aspect of the present invention to provide an improved device and method for joint correction, where the device is not left in the body as a foreign object, but rather is bioabsorbed.

It is another object of the present invention to provide a new wedge which is suitable for osteotomy operations, wherein the wedge enables bone growth to take place, whilst simultaneously biodegrading.

### SUMMARY OF THE INVENTION

According to a first aspect of the present invention there is provided a device having the features of claim 1 and the dependent claims which are appended hereto.

In another embodiment, the invention may also be described as a device for regenerating bone, the device being formed from magnesium or an alloy of magnesium, and comprising a coating layer of Calcium phosphate wherein the Calcium Phosphate is not hydroxyapatite (HA). Hydroxyapatite is a naturally occurring mineral form of calcium apatite with the formula Ca₅(PO₄)₃, often written Ca₁₀(PO₄)₆(OH)₂ to denote that the crystal unit cell comprises two entities.

The calcium phosphate may comprise any one of the group consisting of: octacalcium phosphate (OCP), dicalcium phosphate dihydrate (DCPD); and derivative(s) of OCP, and derivative(s) of DCPD.

The device may comprise an upper surface and a lower surface, wherein the upper surface and the lower surface converge towards a first end and diverge towards a second end to form a pre-determined geometry.

The pre-determined geometry may be one of a wedge, a nail, a screw, a fastener, a plate, or a wire.

The upper and lower surfaces may be for mating against a bone surface inside a human or animal body.

The device may comprises at least one channel which extends from the upper surface to the lower surface, defining a conduit for fluid communication between a first region of the device and a second region of the device and wherein the at least one channel may facilitate bone growth.

The wedge may comprise a plurality of channels extending from the upper surface to the lower surface, wherein the plurality of channels may be arranged in a pre-determined pattern.

The pattern may comprise a plurality of rows arranged in a generally lateral direction between the first end and the second end.

The diameter of the channels in each row may generally increase from the first end towards the second end.

The diameter of the channels may range from about 0.5 mm-4 mm, preferably around 1 mm-3 mm.

The at least one channel or the plurality of channels may be generally cylindrical. The channels may be any cross-sectional shape, to suit any particular application.

The at least one channel or the plurality of channels may be coated with a calcium phosphate material, preferably, comprising octacalcium phosphate (OCP) or DCPD.

The wedge may comprise at least one protrusion for mating into a bone, wherein the at least one protrusion may be located on the upper surface and/or the lower surface of the body.

The at least one protrusion may comprise a channel suitable for receiving a fixing.

The at least one protrusion may be generally a truncated pyramid shape.

The wedge may comprise a fixing plate for receiving fixings, wherein the fixing plate is attached to the body.

The first end may have a thickness which is less than the thickness of the second end.

The wedge may be suitable for one of an distal tibial osteotomy, a high tibial osteotomy, a Evans wedge osteotomy, a Cotton wedge osteotomy or a distal radius wedge osteotomy.

According to another embodiment, there is described a method of manufacturing a device for bone regeneration, the method comprising:
providing a device formed of a base material of magnesium or magnesium alloy;
coating the device with a coating of Calcium phosphate wherein the Calcium Phosphate is not hydroxyapatite (HA).

The calcium phosphate may comprise any one of the group consisting of: octacalcium phosphate (OCP), dicalcium phosphate dihydrate (DCPD); and derivative(s) of OCP, and derivative(s) of DCPD.

The method may comprises the step of: immersing the device in a solution for depositing a coating of calcium phosphate on the device.

Generally, the invention relates to a device which can be inserted into the human or animal body, and can be used in orthopaedic surgeries.

The device may be a wedge and is made from an octacalcium phosphate (OCP) coated magnesium alloy. The base material of the device may be pure magnesium, or it may be a magnesium alloy.

The base material may be composed of magnesium (95-99%), manganese (0.01-5%), zinc (0.01-5%), calcium (0.01-5%), and phosphate (0.01-5%).

Magnesium or magnesium alloys have similar mechanical properties to bone, which is essential in providing strength and stability for osteotomy procedures.

Magnesium is also highly biocompatible to the human body mitigating risks of an inflammatory response. The advantage of using magnesium as an implant is its degradation properties magnesium and magnesium alloys degrade in in vivo surroundings. The rate of degradation is managed by coating with OCP to maximise the strength and stability of the physical wedge construct in supporting the bone, while degrading at similar rates to the healing bone.

Increasing the thickness of the OCP coating increases the total degradation time of the magnesium component. Conversely, decreasing the thickness of the OCP coating decreases the total degradation time of the magnesium component.

Generally speaking, the present invention relates to a OCP coated magnesium or magnesium alloy wedge for osteotomy.

To maximise the healing potential of the bone and the stability of the implant, the wedges have an pattern of channels for this function. The channels are located on the thicker parts of the wedges and travel from one end to the other end creating tunnel like structures. As osteoblasts cannot ‘jump’ more than 3 mm distance for healing purposes, the longitudinal channels facilitate this process by their locating at the thicker parts of the wedge. The channels are also coated with OCP as OCP promotes the healing of bone, thus promoting bone growth through the channels.

The size of the channels are relative to the space they occupy on the wedge. For example, where the channel has to occupy a wider area and thicker area, the channel will have a larger diameter. For example in a square unit of 3×3 mm a channel may have a diameter of 1.2 mm; a square unit of 5×5 mm a channel may have a diameter of 2 mm and a square unit of 6.25 mm×6.25 mm a channel may have a diameter of 2.5 mm diameter.

The sizes of the units and diameters of channels are selected to maximise bone growth and healing while maintaining maximum stability in the line of load bearing. The larger the channel diameter, the more bone growth potential however mechanical stability would be compromised (and vice versa).

The channels carry the same function in all of the wedge designs and are the key promoters of maximising the function of OCP and magnesium together into a unit.

The wedges are designed in the correct anatomical shape to fit the area in the body they are treating. The wedges are anatomically designed to treat osteotomies and maximise the replacement of natural bone through OCP function of bone healing and magnesium implant stability to support the bone healing and maintain stability during the magnesium degradation process.

The wedges may be fit with protrusions to prevent slippage, a common occurrence of current wedge design due to the slanted shape and movement in the body. These may also be coated with OCP. Some phalanges are required to be of a small size where they act like a small stopper from slipping. Some wedges are required to be of a larger size to ensure stability and have screw holes placed through them for stability with screw fixation, such as the wedge for the Evans osteotomy procedure.

Some wedges are fit with a plate positioned at the back for better fixation of the wedge to this particular anatomical site. Holes in the plates are present for screw fixation to secure plate to bone and wedge.

### BRIEF DESCRIPTION OF THE DRAWINGS

Embodiments of the present invention will now be described, by way of example only, with reference to the following figures:

FIG. 1 is a graph showing the relationship between magnesium degradation and bone healing;

FIG. 2 is an elevation view of a deformed ankle joint;

FIG. 3 is an elevation view of the ankle joint of FIG. 2 where the tibia has been cut and distracted, and also indicating where a wedge would be inserted;

FIG. 4 is an elevation view of the ankle joint of FIGS. 2 and 3, wherein the wedge has been inserted into the correct anatomical position;

FIG. 5 is an elevation view of the ankle joint of FIG. 4, further showing the process of bone cell travel through the longitudinal channels;

FIG. 6 is an elevation view of the ankle joint in FIG. 5, showing the joint after degradation of the wedge has taken place;

FIG. 7 is a plan view of a medical distal tibial wedge according to an embodiment of the present invention;

FIG. 8 is a perspective view of the medical wedge in FIG. 7;

FIG. 9 is a side elevation view of the wedge in FIGS. 7 and 8;

FIG. 10 is a plan view of a medical high tibial wedge according to an embodiment of the present invention;

FIG. 11 is a perspective view of the medical wedge in FIG. 10;

FIG. 12 is a side elevation view of the wedge in FIGS. 10 and 11;

FIG. 13 is a plan view of a medical Evans wedge according to an embodiment of the present invention;

FIG. 14 is a perspective view of the medical wedge in FIG. 13;

FIG. 15 is a side elevation view of the wedge in FIGS. 13 and 14;

FIG. 16 is a plan view of a medical Cotton wedge according to an embodiment of the present invention;

FIG. 17 is a perspective view of the medical wedge in FIG. 16;

FIG. 18 is a side elevation view of the wedge in FIGS. 16 and 17;

FIG. 19 is a plan view of a medical distal radius wedge according to an embodiment of the present invention;

FIG. 20 is a perspective view of the medical wedge in FIG. 19;

FIG. 21 is a side elevation view of the wedge in FIGS. 19 and 20;

FIG. 22 is a plan view of a medical scaphoid wedge according to an embodiment of the present invention;

FIG. 23 is a perspective view of the medical wedge in FIG. 22;

FIG. 24 is a side elevation view of the wedge in FIGS. 22 and 23; and

FIG. 25 is an illustrative example of a coating method for coating a magnesium or magnesium alloy.

### DETAILED DESCRIPTION

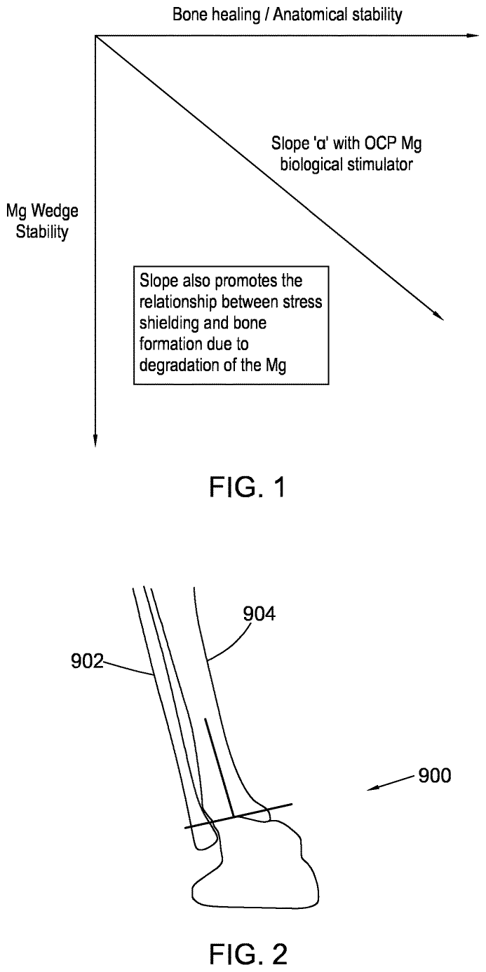

FIG. 1 is a graph showing the relationship between magnesium degradation and bone healing. As shown, bone healing or anatomical stability is shown on the X-axis, and the wedge stability is shown on the Y-axis. The diagonal line on the graph shows that there is a directly proportional relationship between the stability of the bone and the wedge stability. In summary, as the wedge degrades the bone heals.

FIGS. 2-6 show a typical process of a surgical process to correct a joint deformity with osteotomy. Although the invention will be described herein in relation to a wedge, this should not be construed to be limiting. The device may be any device which is to be left inside a human or animal body after an osteo surgery has taken place. The device may be a screw (headed or headless), a fastener of any sort, a Kirschner wire, a pin, a nail, a plate etc.

FIG. 2 is an elevation view of a deformed ankle joint 900. The bones above the ankle, the fibula 902 on the left, and the tibia 904 on the right, are displaced to the left of the image, creating a misaligned ankle joint 900. This is illustrated by the perpendicular ‘T’ overlay, which clearly illustrates the vertical offset of the tibia 904.

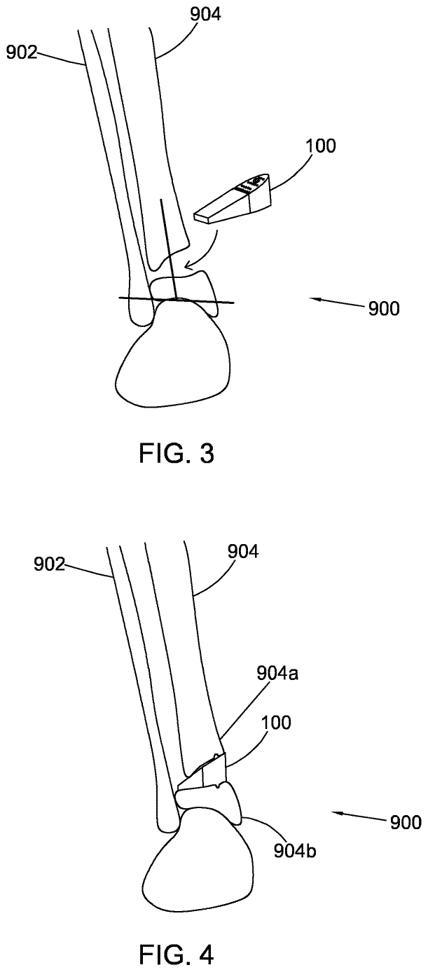

FIG. 3 is an elevation view of the ankle joint 900 of FIG. 2 where the tibia 904 has been cut and distracted. The joint 900 has been realigned, where it awaits the insertion of a wedge 100. A wedge 100 according to the invention is shown in the Figure, illustrating a typical location where it would be inserted into the distracted tibia 904.

FIG. 4 is an elevation view of the ankle joint 900 of FIGS. 2 and 3, showing the wedge 100 in the inserted position. The wedge 100 is located in the correct anatomical position for optimum bone healing and support of the joint 900. The protrusions 120 (shown in more detail in further figures) located on the upper surface 102 and lower surface 104 of the wedge 100 lodge into the upper section 904a and the lower section 904b of the distracted tibia 904. This is to increase friction, and to reduce the likelihood of dislodgement or movement between the wedge 100 and the bone sections 904a/b.

The magnesium base material of the wedge 100 offers strength and support to the bone structures for the initial healing weeks, whilst the OCP coating causes delayed degradation of the wedge. The thickness of the OCP coating can be adjusted to provide a bespoke degradation rate, depending on the patient and location of the wedge.

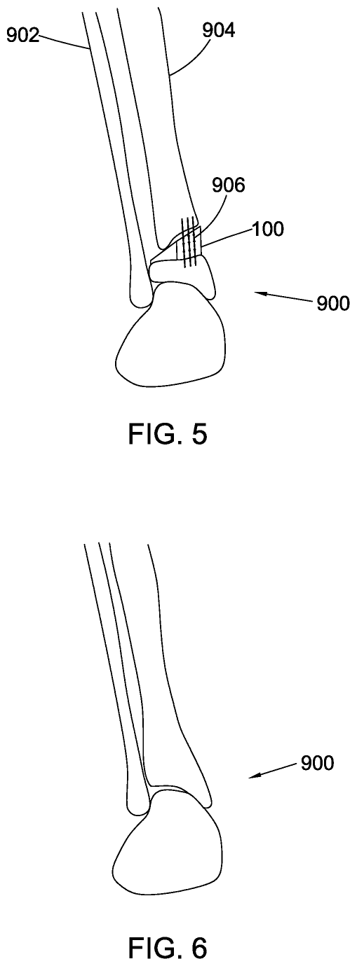

FIG. 5 is an elevation view of the ankle joint 900 of FIG. 4, further showing the process of bone cell travel 906 through channels located in the wedge. The bone healing commences simultaneously with the wedge 100 degradation, thus providing a secure support for the bone 904 to heal, whilst the wedge 100 degrades.

The OCP coated channels bridge the gap for bone cells to travel along, thus creating bone within the channels. Arrows 906 depict this process of bone growth within the channels.

The wedge 100 in the example only comprises channels at the thick end of the wedge (right side in image), as the thin end (left side in image) is thin enough for the bone to heal without the assistance of channels.

FIG. 6 is an elevation view of the ankle joint 900 in FIG. 5, showing the joint 900 after degradation of the wedge has taken place. The bone and joint structure is shown as being fully healed, and the patient can continue with their lives without the drawbacks of having any foreign bodies with their leg.

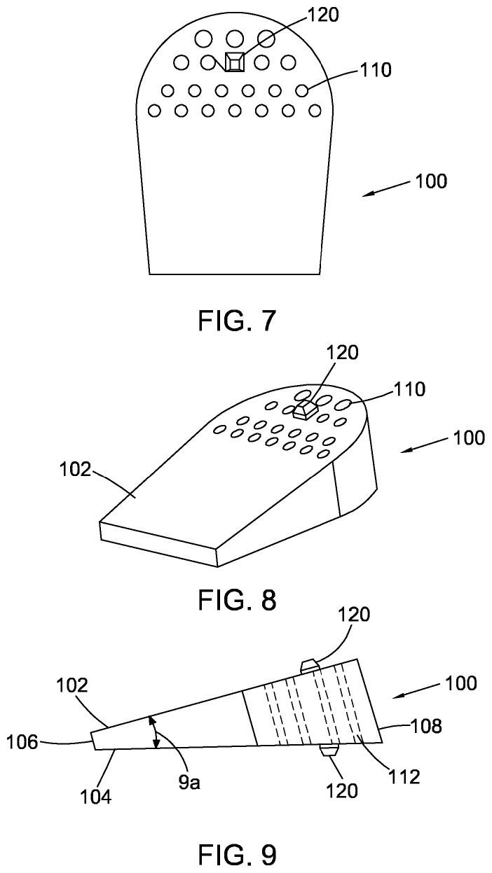

FIG. 7 is a plan view of a medical distal tibial wedge 100 according to an embodiment of the present invention, FIG. 8 is a perspective view of the medical wedge 100 in FIG. 7; and FIG. 9 is a side elevation view of the wedge 100 in FIGS. 7 and 8.

The present invention will be described with reference to an exemplary wedge, shown in the attached Figures as wedge 100. It is to be understood that wedge 100 is a preferred example but is merely one of the various shaped wedges 100 that are provided by this disclosure. The particular exemplary shape shown in FIGS. 7-9 is suitable for inserting in the lower portion of the tibial bone of the leg. It is to be understood that other shapes of wedges or other shapes in other forms of the device of the present disclosure can be provided, within the scope of the present disclosure, configured for or suitable for use at or in other bone regions including at or in bone joints.

Referring to the exemplary wedge 100, the base material of the wedge 100 is made from magnesium, or a magnesium alloy. The base material is coated with octacalcium phosphate (OCP), which functions to protect the magnesium from degrading too quickly. The thickness of the OCP coating determines the degradation rate of the wedge 100.

The wedge as shown comprises an upper surface 102 and a lower surface 104. Upper and lower should not be construed to be limiting to any particular orientation. The terms are used merely for ease of reference.

The upper surface 102 and the lower surface 104 converge towards a first end 106, and diverge towards a second end 108. The thickness of the first end 106 is shown to be less than the thickness of the second end 108. The upper surface 102 and the lower surface 104 together create the wedge shape of the wedge 100, allowing the wedge to be driven into the space between the patient's bone.

The wedge angle aa between the upper surface 102 and the lower surface 104 may vary from the angle aa as shown. The angle aa as shown in FIGS. 8 and 9 should be taken to be one example of a typical wedge 100. The angle aa of the wedge 100 may be dictated by the geometry of the patients deformity for example. The angle aa may be any angle within the range of 0-90 degrees.

The wedge 100 also comprises a plurality of apertures 110. The apertures 110 extend from the upper surface 102 through to the lower surface 104, forming channels 112. The channels 112 are shown in FIG. 9 extending through the wedge 100. The channels 112 as shown are cylinders with a uniform cross sectional area.

The channels 112 may have any suitable cross sectional area, such as square, a triangular or rectangular.

The channels 112 permit bone growth between the upper bone surface and the lower bone surface. The channels 112 are coated in an OCP coating, in the same manner as the rest of the wedge 100.

The channels 112 are arranged in a pattern as shown. The pattern is comprised of four rows, each row being arranged in a lateral orientation between the first end 106 and the second end 108.

The diameter of the apertures 110 is the same in each row, however the diameter of each aperture 110 increases with each row from the first end 106 to the second end 108. The diameter of the channels 112 is therefore larger towards the thicker second end 108, than the diameters of the channels 112 towards the thinner end 106. This is to allow more bone growth where the wedge 100 is thicker. The larger diameter channels 112 permit bone growth to occur at a higher rate than the smaller diameter channels 112.

Osteoblasts cannot ‘jump’ more than 3 mm distance for bone healing or forming purposes, and so the channels 112 facilitate this process by being located at the thicker parts of the wedge 100, where the thickness of the wedge is greater than 3 mm.

If the channel diameter is too narrow, the bone growths could block the channel, however if they are too large, the device may be too weak for the application. The channel diameter therefore needs to be regulated according to the design of the device.

Although the pattern is shown in with four rows of apertures 110, there may be any number of rows. There may be any number of columns of apertures 110 within the pattern. The pattern may be regular (as shown), or may be irregular—i.e. where the rows and columns are not so clearly distinguishable.

In general, in all of the embodiments of the invention, the channels 112 nearer the thicker second end 108 of the wedge 100 will have larger diameters than the channels 112 which are nearer the thinner first end 106 of the wedge 100. Although this is true of the embodiments shown, it should not be construed to be a limiting feature.

The diameter of the channels 112 may range between 0.5 mm and 4 mm. Preferably, the diameter of the channels may be between 1 mm and 3 mm. The diameter of the channels 112 will depend on the thickness and size of the wedge 100. Thicker wedges 100 in general will need larger diameter channels 112. The sizes of the wedges 100 and the diameters of the channels 112 are selected to maximise bone growth and healing, whilst maintaining a high level of stability in the line of load bearing. Large diameter channels 112 has the highest bone growth potential, however overly large channels 112 can compromise mechanical bone stability (and vice versa).

The channels 112 may also be coated with octacalcium phosphate (OCP), in a similar manner to the base material of the remainder of the wedge 100. The OCP coating promotes the healing of the bone, thus promoting bone growth through the channels 112.

Protrusions 120 are shown in FIGS. 7-9, with one protrusion 120 extending from the upper surface 102 and another protrusion extending from the lower surface 104. The protrusions 120 are shown to be located within the pattern, however the protrusions 120 may be located anywhere on upper surface 102 or the lower surface 104 of the wedge 100. The protrusions 120 are designed to prevent any slippage between the wedge 100 and the bone structures surrounding it. Slippage is a common occurrence in prior art wedge designs, due to the slanted shape of a wedge and the moving environment within the body.

The protrusions 120 may vary in size from the protrusions 120 shown. Some protrusions 120 are required to be relatively small, to act like a stopper to prevent slippage. Some protrusions 120 require to be a larger size, to ensure stability, and to potentially be suitable for inserting a fixing through them. For example, some protrusions 120 may be large enough for inserting a screw or any suitable fixing through them into the adjacent bone. Some protrusions 120 may comprise an aperture for receiving a screw or other suitable fixing.

The protrusions 120 as shown are shaped as a truncated pyramid, however the protrusions 120 may be any suitable shape for increasing the friction between the bone and the wedge 100.

The remaining Figures are similar to FIGS. 7-9, however the references have been increased by 100 each time, i.e. ‘protrusions 120’ becomes ‘protrusions 220’ etc.

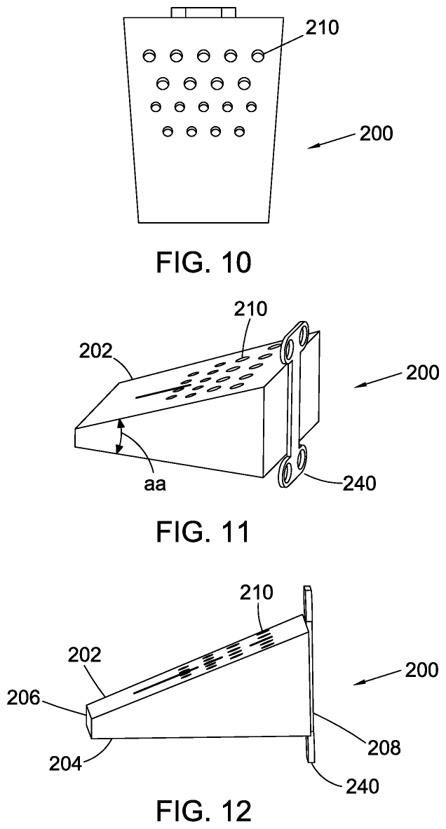

FIG. 10 is a plan view of a medical high tibial wedge 200 according to an embodiment of the present invention, FIG. 11 is a perspective view of the medical wedge 200 in FIG. 10, and FIG. 12 is a side elevation view of the wedge 200 in FIGS. 10 and 11.

The high tibial wedge 200 is similar to the distal tibial wedge 100, however the wedge 200 is more suited for inserting closer to the knee of the patient, as opposed to the ankle.

In this embodiment, the wedge 200 comprises apertures 210 which increase in diameter as the thickness of the wedge 200 increases, as in the first embodiment. The wedge 200 comprises a first end 206 which is thinner than a second end 208.

The apertures 210 are arranged in four rows, and are only located on the thickest part of the wedge 200. Once again, the apertures 210 extend fully through from the upper surface 202 to the lower surface 204 of the wedge 200.

The wedge 200 does not comprise any protrusions, but instead comprises a fixing plate 240 for receiving fixings. The fixing plate 240 is attached onto the second end 208 of the wedge. A fixing plate 240 such as the plate shown, may have several holes to receive screws, and may be located anywhere on the wedge 200.

The fixing plate 240 and fixings may be made from any suitable material. The fixing plate 240 and fixings may be made from a similar material as the wedge 200, i.e. a magnesium base material, coated with OCP.

The fixing plate 240 is used to provide extra security, and provides a stronger connection between the bone and the wedge 200. This is particularly important in locations of large stress, such as a high tibial osteotomy. The fixing plate 240 may be attached onto the wedge 200 by any suitable means, such as an adhesive or screw fixings for example.

Once again, the angle aa between the upper surface 202 and the lower surface 204 should be taken as an example only, as the wedges 200 may have any range of angle aa.

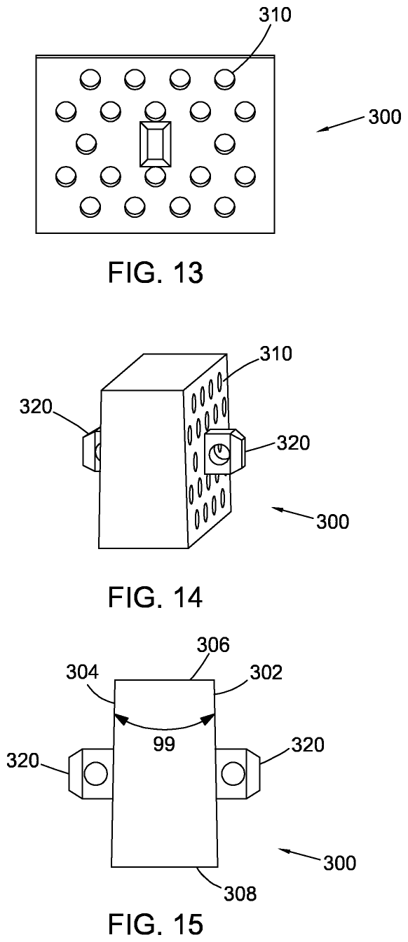

FIG. 13 is a plan view of a medical Evans wedge 300 according to an embodiment of the present invention, FIG. 14 is a perspective view of the medical wedge 300 in FIG. 13, and FIG. 15 is a side elevation view of the wedge 300 in FIGS. 13 and 14.

This wedge 300 is a typical wedge which would be used in an Evans procedure for flat foot correction. The wedge 300 shape is designed to match calcaneus anatomy.

The wedge 300 comprises channels 310 as the previous wedges, however these channels are all the same diameter. This is because the wedge angle aa is so low. There is only a slight taper to the wedge 300, due to the location in the foot. The diameter of the channels 310 and channels are optimised for bone growth.

The protrusions 320 on this wedge 300 are relatively large and have fixing holes in them. The large protrusions 320 help to avoid dislodgement of the wedge. The fixings are required for Evans wedges 300 because they are in an area of high weight bearing.

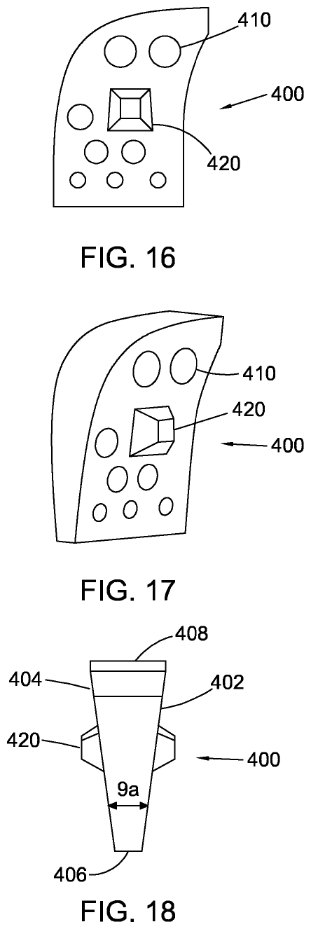

FIG. 16 is a plan view of a medical Cotton wedge 400 according to an embodiment of the present invention, FIG. 17 is a perspective view of the medical wedge 400 in FIG. 16, and FIG. 18 is a side elevation view of the wedge 400 in FIGS. 16 and 17.

The cotton wedge 400 is typically used to correct the arch of the foot. As in previous embodiments, the diameter of the apertures 410 varies with the thickness of the wedge 400. The apertures 410 extend through the thickness of the wedge, forming channels.

The protrusions 420 which extend from the upper surface 402 and the lower surface 404 are generally shaped like a truncated pyramid. The protrusions 420 are required to prevent movement of the wedge 400 during movement of the foot. The protrusions 420 shown do not have any holes for receiving a fixing, however protrusions with holes may easily be used in conjunction with this embodiment.

The Cotton wedge 400 is shaped to accurately fit in the anatomy of the medial cuneiform.

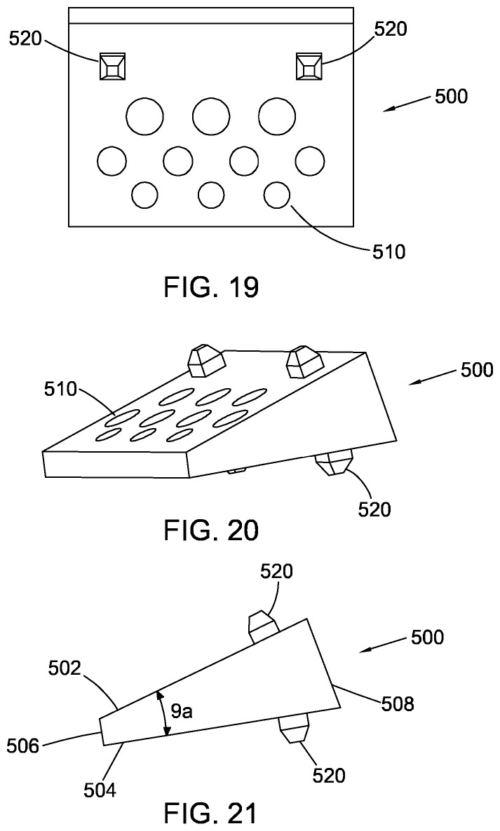

FIG. 19 is a plan view of a medical distal radius wedge 500 according to an embodiment of the present invention, FIG. 20 is a perspective view of the medical wedge 500 in FIG. 19, and FIG. 21 is a side elevation view of the wedge 500 in FIGS. 19 and 20.

The distal radius wedge 500 is a typical wedge which would be used in correcting alignment issues in the wrist. The wedge comprises channels 520 which vary in size depending on which row they are in. Again, the diameter of the channels 520 increases towards the second end 508, thus creating larger diameter channels closer to the second end 508.

Other modifications and features are similar to the other embodiments previously described. For example. A fixing plate may be used with the distal radius wedge 500 if required.

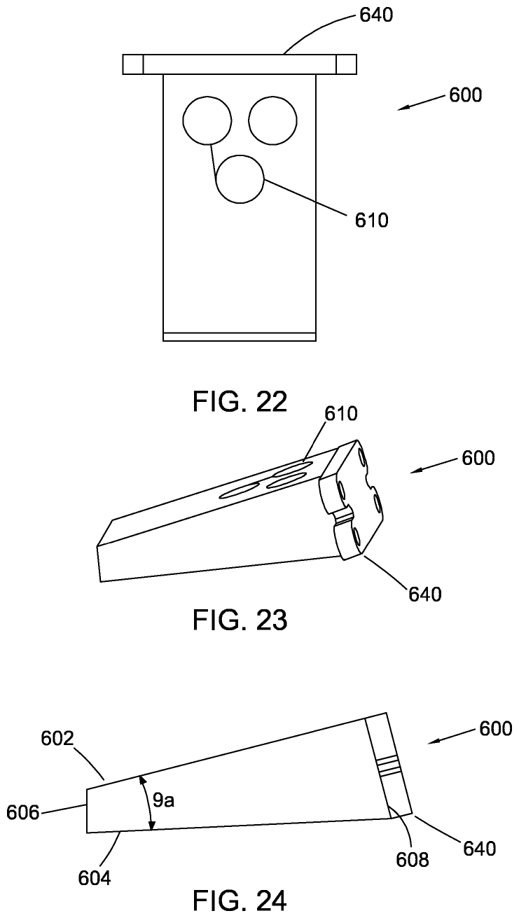

FIG. 22 is a plan view of a medical scaphoid wedge 600 according to an embodiment of the present invention, FIG. 23 is a perspective view of the medical wedge 600 in FIG. 22, and FIG. 24 is a side elevation view of the wedge 600 in FIGS. 22 and 23.

A scaphoid wedge 600 as shown in FIGS. 22-24 is designed to be used in the hand of a patient. This particular example uses two rows of channels 610, forming three distinct channels. The channels extend throughout the width of the wedge 600.

The wedge 600 does not comprise any protrusions, but comprises a fixing plate 640. Once again, the angle aa and general dimensions of the wedge 600 should not be construed to be limiting.

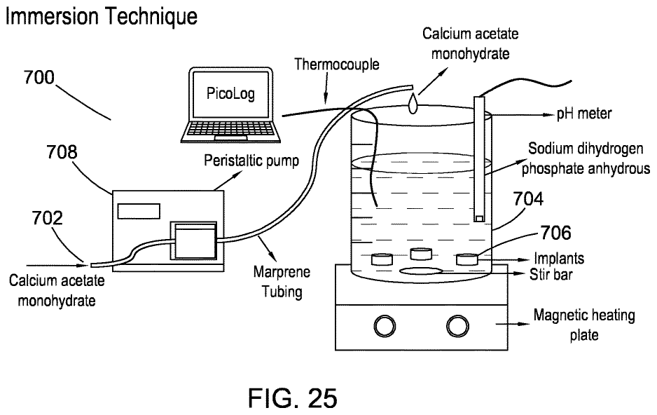

FIG. 25 is an illustrative example of a coating method 700 for coating a magnesium or magnesium alloy. The devices according to the present invention may be coated in this manner.

The coating method 700 introduces a calcium solution 702 into a container 704 which contains the magnesium devices 706 to be coated. The calcium solution 702 may be pumped into the container 704 via a pump 708. The container 704 may contain a solution which contains sodium dihydrogen phosphate anhydrous. The calcium solution 702 may contain calcium acetate monohydrate.

The solution in the container 704 may be stirred and heated to increase the rate of coating. The temperature of the solution in the container 704 may be held at around 50 degrees Celsius. The pH of the solution may be between 3 and 7.

Other known methods in the art may be used to coat the magnesium or magnesium alloy devices in an OCP coating according to the invention.

The devices previously described may be formed any suitable way. For example, the device may be cast moulded. The device may then be cold drilled to form the channels. An alternative method of manufacture is that the device is 3D printed. This avoids the requirement for any drilling, and also enables the channels to have any type of cross sectional shape.

Any of the features in any of the embodiments may be combined in any manner with any of the embodiments shown.

Whilst specific embodiments of the present invention have been described above, it will be appreciated that departures from the described embodiments may still fall within the scope of the present invention.

1. A device for regenerating bone, the device being formed from magnesium or an alloy of magnesium, and comprising a coating layer of Calcium phosphate wherein the Calcium Phosphate is not hydroxyapatite (HA).

2. A device according to claim 1 wherein the calcium phosphate comprises any one of the group consisting of: octacalcium phosphate (OCP), dicalcium phosphate dihydrate (DCPD); and derivative(s) of OCP, and derivative(s) of DCPD.

3. A device according to claim 1 wherein the device comprises an upper surface and a lower surface, wherein the upper surface and the lower surface converge towards a first end and diverge towards a second end to form a pre-determined geometry.

4. A device according to claim 3 wherein the pre-determined geometry is one of a wedge, a nail, a screw, a fastener, a plate, or a wire.

5. A device according to claim 3, wherein the upper and lower surfaces are for mating against a bone surface inside a human or animal body.

6. A device as claimed in any preceding claim wherein the device comprises: at least one channel which extends from the upper surface to the lower surface, defining a conduit for fluid communication between a first region of the device and a second region of the device and wherein the at least one channel facilitates bone growth.

7. A device according to claim 6 wherein the wedge comprises a plurality of channels extending from the upper surface to the lower surface, wherein the plurality of channels are arranged in a pre-determined pattern.

8. A device according to claim 7, wherein the pattern comprises a plurality of rows arranged in a generally lateral direction between the first end and the second end.

9. A device according to either claims 7 or 8, wherein the diameter of the channels in each row generally increases from the first end towards the second end.

10. A device according to any of claims 6 to 9, wherein the diameter of the channels ranges from about 0.5 mm-4 mm, preferably around 1 mm-3 mm.

11. A device according to any preceding claim, wherein the at least one channel or the plurality of channels is/are generally cylindrical.

12. A device according to any preceding claim, wherein the at least one channel or the plurality of channels is coated with a calcium phosphate material, preferably, comprising octacalcium phosphate (OCP) or DCPD.

13. A device according to any preceding claim wherein the wedge comprises at least one protrusion for mating into a bone, wherein the at least one protrusion is located on the upper surface and/or the lower surface of the body.

14. A device according to claim 13, wherein the at least one protrusion comprises an channel suitable for receiving a fixing.

15. A device according to one of claims 13 or 14, wherein the at least one protrusion is generally a truncated pyramid shape.

16. A device according to any one of claims 2 to 15, wherein the wedge comprises a fixing plate for receiving fixings, wherein the fixing plate is attached to the body.

17. A device according to any preceding claim, wherein the first end has a thickness which is less than the thickness of the second end.

18. A device according to any preceding claim, wherein the wedge is suitable for one of an distal tibial osteotomy, a high tibial osteotomy, a Evans wedge osteotomy, a Cotton wedge osteotomy or a distal radius wedge osteotomy.

19. A method of manufacturing a device for bone regeneration, the method comprising:
providing a device formed of a base material of magnesium or magnesium alloy;
coating the device with a coating of Calcium phosphate wherein the Calcium Phosphate is not hydroxyapatite (HA).

20. A method as claimed in claim 19, wherein the calcium phosphate comprises any one of the group consisting of: octacalcium phosphate (OCP), dicalcium phosphate dihydrate (DCPD); and derivative(s) of OCP, and derivative(s) of DCPD.

21. A method according to claim 19, wherein the method comprises the steps of:
Immersing the device in a solution for depositing a coating of calcium phosphate on the device.
